(19) United States
(12) Patent Application Publication
(10) Pub. No.: US 2024/0034990 A1
(43) Pub. Date: Feb. 1, 2024

(54) SPERM SORTING DEVICE

(71) Applicant: Bonraybio Co., Ltd., Taichung City (TW)

(72) Inventors: Cheng-Teng Hsu, Taichung City (TW); Fang-Zong Wang, Miaoli City (TW); Han-Yuan Gao, Changhua City (TW)

(21) Appl. No.: 18/455,458

(22) Filed: Aug. 24, 2023

Related U.S. Application Data
(63) Continuation-in-part of application No. 17/815,484, filed on Jul. 27, 2022.

(30) Foreign Application Priority Data
Aug. 24, 2022 (TW) 111209204

(51) Int. Cl. C12N 5/071

(52) U.S. Cl. CPC C12N 5/0612

(57) ABSTRACT

A sperm sorting device suitable for selectively isolating a plurality of motile sperms from a semen specimen includes a base whose bottom wall and surrounding wall define a receiving space, and a sorter securely coupled to the base and including a mounting member and a sorting filter. The mounting member has a sleeve located in the receiving space and an injection portion located at the bottom end of the sleeve and having an injection hole. The sorting filter is fixed on the sleeve and, together with the injection portion and the sleeve, defines a sorting chamber with an open top end. The sorting filter, the bottom wall, and the surrounding wall define a specimen chamber for receiving the semen specimen. The injection hole brings the specimen chamber and the sorting chamber into communication vertically, so the semen specimen can be injected through the injection hole into the specimen chamber rapidly.

### CROSS-REFERENCE TO RELATED APPLICATIONS

This application is a continuation-in-part of U.S. patent application Ser. No. 17/815,484 entitled “SPERM SORTING DEVICE AND METHOD” (Attorney Docket No. 120390-8006.US01) filed Jul. 27, 2022, which is entitled to the benefit of and/or the right of priority to Taiwanese Application No. 111110406, entitled “SPERM SORTING DEVICE AND METHOD,” filed Mar. 21, 2022, and Taiwanese Application No. 110128419, entitled “SPERM SORTING DEVICE AND METHOD,” filed on Aug. 2, 2021; this application is also entitled to the benefit of and/or the right of priority to Taiwanese Application No. 111209204, entitled “SPERM SORTING APPARATUS,” filed on Aug. 24, 2022; all of the aforementioned applications are hereby incorporated by reference in their entireties for all purposes.

### BACKGROUND OF THE INVENTION

### 1. Technical Field

The present invention relates to a tool designed specifically for medical use. More particularly, the invention relates to a sperm sorting device.

### 2. Description of Related Art

Referring to FIG. 1, the sperm sorting system 10 disclosed in U.S. Pat. No. 10,422,737 B2 includes a housing 12 formed with an inlet 11, a collection chamber 13 provided in the housing 12 and located beside the inlet 11, a microfluidic chip 14 mounted on the bottom side of the housing 12, a flow path 15 extending from the inlet 11 to the collection chamber 13, and a filter 16 provided in the collection chamber 13. The flow path 15 has a (vertical) gap between 100 nanometers and 500 micrometers. The collection chamber 13 includes a bottom chamber 17 adjacent to the microfluidic chip 14 and a top chamber 18 located on top of the bottom chamber 17 and farther away from the microfluidic chip 14 than is the bottom chamber 17. The bottom chamber 17 is configured to collect the semen sample injected through the inlet 11 into the flow path 15 so that motile sperms in the semen sample can swim upward through the filter 16 into the top chamber 18 and thus become selectively isolated from the semen sample.

While the sperm sorting system 10 can sort sperms, it has the following drawbacks:
(1) The path from the inlet 11 to the bottom chamber 17 is relatively long and has two turns, so when a user injects a semen sample through the inlet 11 into the narrow flow path 15 with a syringe, it is required that the user apply a force to the syringe continuously in order to generate a pressure high enough to push the semen sample through the narrow flow path 15 into the bottom chamber 17. The aforesaid operation, however, is laborious and time-consuming.
(2) The flow path 15 must be provided, which nevertheless leads to a relatively complicated overall structure and hence a relatively high production cost.
(3) The flow path 15 extends horizontally between the inlet 11 and the bottom chamber 17 and thus increases the diameter of the sperm sorting system 10.

### BRIEF SUMMARY OF THE INVENTION

One objective of the present invention is to provide a sperm sorting device that can be operated with less time and effort, that has a simpler overall structure, and that incurs a lower production cost than its prior art counterparts.

The present invention provides a sperm sorting device suitable for selectively isolating a plurality of motile sperms from a semen specimen. The sperm sorting device includes a base and a sorter. The base includes a bottom wall and a surrounding wall that extends from the periphery of the bottom wall toward the top end of the base and that surrounds an axis. The bottom wall and the surrounding wall jointly define a receiving space that has an opening. The sorter is securely coupled to the base and includes a mounting member and a sorting filter. The mounting member has a sleeve, a liquid drawing ring, and an injection portion. The sleeve surrounds the axis and is placed in the receiving space. The liquid drawing ring extends from the bottom end of the sleeve toward the axis. The injection portion is provided on the liquid drawing ring. Moreover, the injection portion is penetrated by an injection hole along a direction parallel to the axis, and the sleeve has an inner sleeve surface and an outer sleeve surface that surrounds the inner sleeve surface. The sorting filter is fixed on the sleeve in such a way that the sorting filter, the liquid drawing ring, and the inner sleeve surface jointly define a sorting chamber that has an open top end. The sorting filter has a plurality of micropores through which the motile sperms can pass. The sorting filter, the bottom wall, and the surrounding wall jointly define a specimen chamber for receiving the semen specimen. The injection hole brings the specimen chamber and the sorting chamber into communication with each other vertically.

The sperm sorting device has the following effects: The liquid drawing ring is provided with the injection portion in order for the specimen chamber and the sorting chamber to communicate with each other vertically in a direct manner through the injection hole, thereby allowing the semen specimen to be injected through the injection hole into the specimen chamber rapidly. The sperm sorting device, therefore, can be operated with less time and effort, has a simpler overall structure, and incurs a lower production cost than its prior art counterparts.

The present invention also provides a sperm sorting device that is suitable for selectively isolating a plurality of motile sperms from a semen specimen and that has the following features. The sperm sorting device includes a base and a sorter. The base includes a bottom wall and a surrounding wall that extends from the periphery of the bottom wall toward the top end of the base and that surrounds an axis. The bottom wall and the surrounding wall jointly define a receiving space that has an opening. The sorter is securely coupled to the base and includes a mounting member and a sorting filter. The mounting member has a sleeve and an injection portion. The sleeve surrounds the axis and is placed in the receiving space. The injection portion extends from the bottom end of the sleeve toward the axis. Moreover, the injection portion is penetrated by an injection hole along a direction parallel to the axis, and the sleeve has an inner sleeve surface and an outer sleeve surface that surrounds the inner sleeve surface. The sorting filter is fixed on the sleeve in such a way that the sorting filter, the injection portion, and the inner sleeve surface jointly define a sorting chamber that has an open top end. The sorting filter has a plurality of micropores through which the motile sperms can pass and a through hole corresponding to the injection hole. The sorting filter, the bottom wall, and the surrounding wall jointly define a specimen chamber for receiving the semen specimen. The injection hole brings the specimen chamber and the sorting chamber into communication with each other vertically.

This sperm sorting device has the following effects: The bottom end of the sleeve is provided with the injection portion in order for the specimen chamber and the sorting chamber to communicate with each other vertically in a direct manner through the injection hole, thereby allowing the semen specimen to be injected through the injection hole into the specimen chamber rapidly. The sperm sorting device, therefore, can be operated with less time and effort, has a simpler overall structure, and incurs a lower production cost than its prior art counterparts.

### BRIEF DESCRIPTION OF THE SEVERAL VIEWS OF THE DRAWINGS

Other features and effects of the present invention can be better understood by referring to the following detailed description in conjunction with the accompanying drawings, in which:

FIG. 1 is an assembled sectional view of the system disclosed in U.S. Pat. No. 10,422,737 B2;

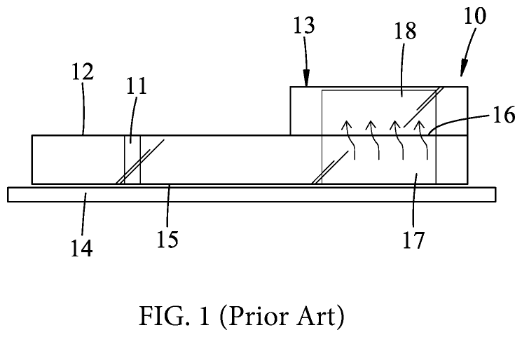

FIG. 2 is an assembled perspective view of the sperm sorting device according to the first embodiment of the invention;

FIG. 3 is a partially exploded perspective view of the first embodiment, showing that the first embodiment includes a base, a sorter, and a cover;

FIG. 4 is an exploded perspective view of the first embodiment;

FIG. 5 is a bottom perspective view of the sorter in the first embodiment;

FIG. 6 is an exploded bottom perspective view of the sorter in the first embodiment;

FIG. 7 is an assembled sectional view of the first embodiment;

FIG. 8 shows a state of use of the first embodiment;

FIG. 9 is a partially exploded perspective view of the sperm sorting device according to the second embodiment of the invention, showing that the second embodiment includes a base, a sorter, and a cover;

FIG. 10 is a bottom perspective view of the sorter in the second embodiment;

FIG. 11 is an exploded bottom perspective view of the sorter in the second embodiment;

FIG. 12 is an assembled sectional view of the second embodiment;

FIG. 13 is a partially exploded perspective view of the sperm sorting device according to the third embodiment of the invention, showing that the third embodiment includes a base, a sorter, and a cover; and

FIG. 14 is an assembled sectional view of the third embodiment.

### DETAILED DESCRIPTION OF THE INVENTION

Before describing the present invention in more detail, it should be pointed out that the terms of relative positions used in the following description, such as “top” and “bottom”, make reference to the directions shown in the drawings and to directions associated with normal use, and that similar elements are denoted by the same reference numeral. In addition, while elements referred to herein as “sleeve” or “ring” are shown in the drawings as circular, it is not required that such a “sleeve” or “ring” be circular.

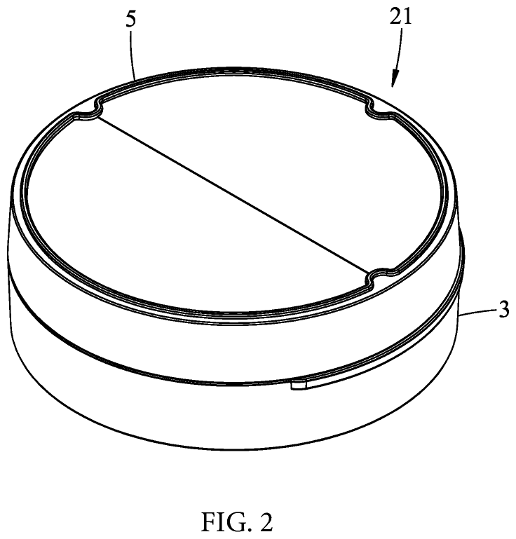

Referring to FIG. 2 to FIG. 4 and FIG. 8, the sperm sorting device 21 according to the first embodiment of the present invention is suitable for selectively isolating a plurality of motile sperms 110 from a semen specimen 100 and includes a base 3, a sorter 4, and a cover 5.

Referring to FIG. 2 to FIG. 4 and FIG. 7, the base 3 includes a bottom wall 31, a surrounding wall 32, a top wall 33, an outer wall 34, and a central protruding block 35. The surrounding wall 32 extends from the periphery of the bottom wall 31 toward the top end of the base 3 and surrounds an axis I. The top wall 33 extends from the top end of the surrounding wall 32 in a direction away from the axis I. The outer wall 34 extends from the outer periphery of the top wall 33 toward the bottom end of the base 3 and surrounds the axis I. The central protruding block 35 extends from a central portion of the bottom wall 31 toward the top end of the base 3 along the axis I, with the diameter of the central protruding block 35 gradually reduced toward the top end of the base 3. The bottom wall 31 and the surrounding wall 32 jointly define a receiving space 36 that has an opening 361.

The central protruding block 35 is a truncated cone-shaped block that is wider at the bottom than at the top and has a truncated cone-shaped surface 351 and a plurality of spaced-apart ribs 352 that are provided on the truncated cone-shaped surface 351 at equal intervals.

In this embodiment, the surrounding wall 32 extends from the bottom wall 31 toward the top end of the base 3 along the axis I, the diameter of the surrounding wall 32 is gradually increased toward the top end of the base 3, and the receiving space 36 is a truncated cone-shaped space that is wider at the top than at the bottom.

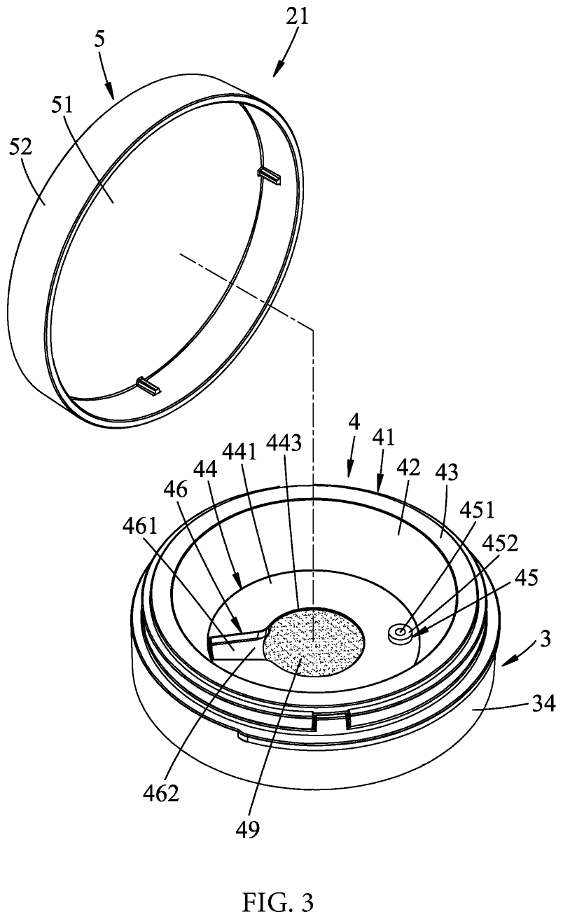

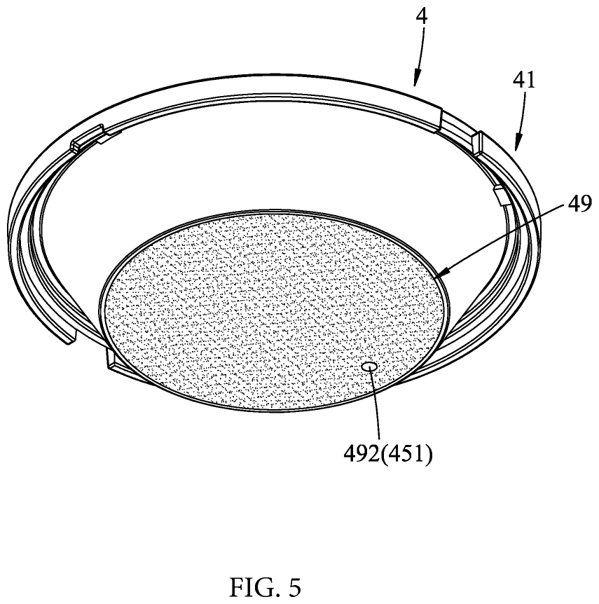

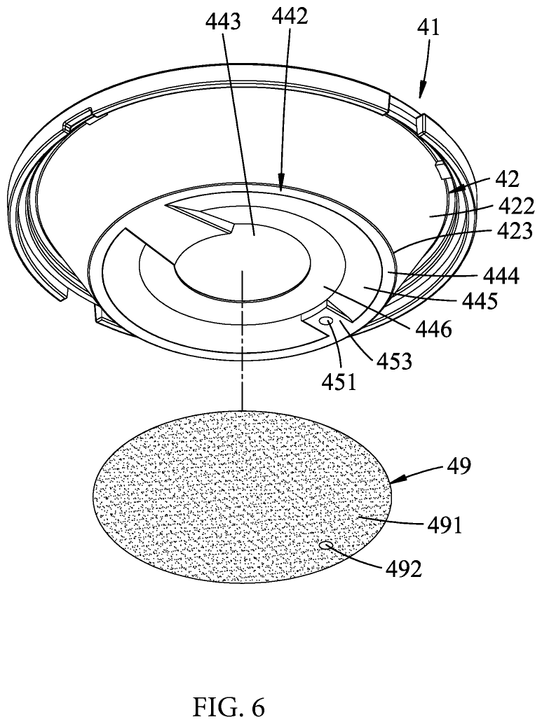

Referring to FIG. 3 to FIG. 8, the sorter 4 is securely coupled to the base 3 and includes a mounting member 41 and a sorting filter 49.

The sorter 4 and the base 3 may be integrally formed as a single structure or be two separate components that are securely coupled to each other to form a single structure.

The mounting member 41 has a sleeve 42, a protruding ring 43, a liquid drawing ring 44, an injection portion 45, and a position-limiting groove 46 for a liquid taking device. The sleeve 42 surrounds the axis I and is placed in the receiving space 36 and securely coupled to the surrounding wall 32. The protruding ring 43 extends from the top end of the sleeve 42 in a direction away from the axis I and is securely coupled to the top wall 33 of the base 3. The liquid drawing ring 44 extends from the bottom end of the sleeve 42 toward the axis I. The injection portion 45 is provided on the liquid drawing ring 44 and extends along a direction parallel to the axis I. The position-limiting groove 46 for a liquid taking device is provided in the liquid drawing ring 44 and has an open top end.

The sleeve 42 has an inner sleeve surface 421, an outer sleeve surface 422 surrounding the inner sleeve surface 421, and a bottom sleeve surface 423 connecting the inner sleeve surface 421 and the outer sleeve surface 422.

The liquid drawing ring 44 has an annular top surface 441, an annular bottom surface 442, and a central hole 443 that brings the annular top surface 441 and the annular bottom surface 442 into communication with each other. The annular bottom surface 442 approaches the annular top surface 441 gradually while extending from the bottom sleeve surface 423 of the sleeve 42 toward the axis I; as a result, the distance between the annular bottom surface 442 and the annular top surface 441 of the liquid drawing ring 44 is gradually reduced as the liquid drawing ring 44 extends from the bottom end of the sleeve 42 toward the axis I.

The annular bottom surface 442 of the liquid drawing ring 44 has an outer extension section 444, a connecting section 445, and an inner extension section 446. The outer extension section 444 extends from the bottom sleeve surface 423 of the sleeve 42 toward the axis I. The connecting section 445 extends from the outer extension section 444 toward the annular top surface 441 and has a diameter that is gradually reduced toward the annular top surface 441. The inner extension section 446 extends from the connecting section 445 toward the axis I. The central hole 443 brings the annular top surface 441 and the inner extension section 446 of the annular bottom surface 442 into communication with each other.

The injection portion 45 connects the annular top surface 441 and the connecting section 445 of the annular bottom surface 442 and has an injection hole 451, a top protruding block 452, and a bottom protruding block 453. The injection hole 451 penetrates the injection portion 45 along a direction parallel to the axis I. The top protruding block 452 is formed on the annular top surface 441 of the liquid drawing ring 44 and surrounds the injection hole 451. The bottom protruding block 453 is formed on the connecting section 445 of the annular bottom surface 442 of the liquid drawing ring 44 and surrounds the injection hole 451.

The position-limiting groove 46 for a liquid taking device extends from the annular top surface 441 toward the annular bottom surface 442 and has a groove bottom portion 461 and a communicating portion 462 that brings the groove bottom portion 461 and the central hole 443 into communication with each other. The width of the position-limiting groove 46 for a liquid taking device may range from 4 μm to 5 mm.

The sorting filter 49 is fixed on the sleeve 42 in such a way that the sorting filter 49, the liquid drawing ring 44, and the inner sleeve surface 421 jointly define a sorting chamber 6 that has an open top end. The sorting filter 49 has a plurality of micropores 491 through which the motile sperms 110 can pass. The micropores 491 may each have a diameter or pore size between 2 and 50 micrometers.

In the first embodiment, the sorting filter 49 is fixed on the outer extension section 444 of the annular bottom surface 442 of the liquid drawing ring 44 and has a through hole 492 corresponding to the injection hole 451.

The sorting filter 49 may be further fixed to the bottom surface of the bottom protruding block 453 and the bottom surface of the groove bottom portion 461 of the position-limiting groove 46 for a liquid taking device to increase the area over which the sorting filter 49 is fixed to the sleeve 42 and to thereby better secure the sorting filter 49 in place.

The sorting filter 49 is located on the bottom side of the central hole 443. The position-limiting groove 46 for a liquid taking device is in communication with the central hole 443 and the sorting filter 49 by way of the communicating portion 462.

Once the sorter 4 is coupled to the base 3, the sleeve 42 of the mounting member 41 is located in the receiving space 36, and the sorting filter 49, the bottom wall 31, and the surrounding wall 32 jointly define a specimen chamber 7 for receiving the semen specimen 100. The specimen chamber 7 and the sorting chamber 6 communicate with each other vertically in a direct manner through the injection hole 451 and the through hole 492.

The cover 5 includes a main portion 51 and a surrounding portion 52. The surrounding portion 52 extends from the periphery of the main portion 51 toward the bottom end of the cover 5, surrounds the axis I, and is configured to rest on the protruding ring 43 and the top wall 33. The cover 5 can be used to close the sorting chamber 6.

In some embodiments, the sorter 4 includes a protruding ring 43 that is located on a periphery of the sorter 4. The protruding ring 43 may include an inner sleeve surface 421 and a central hole 443. The inner sleeve surface 421 may extend toward a bottom of the sorter 4. A sorting filter 49 may span the central hole 443 and filter semen specimen.

The sorter 4 also may also include an injection portion 45 with an injection hole 451 extending therethrough. The injection portion 45 may be configured to join a sorting space above the sorter 4 to a receiving space 36 in the base 3 and may be located above the receiving space 36. The injection hole 451 of the injection portion 45 may align with a through hole 492 in the sorting filter 49. In some embodiments, the sorting filter 49 does not extend over the injection portion 45.

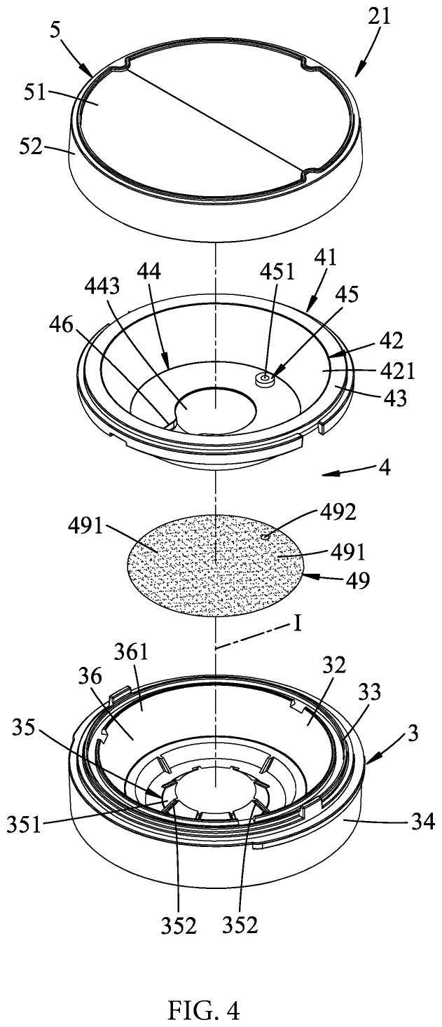

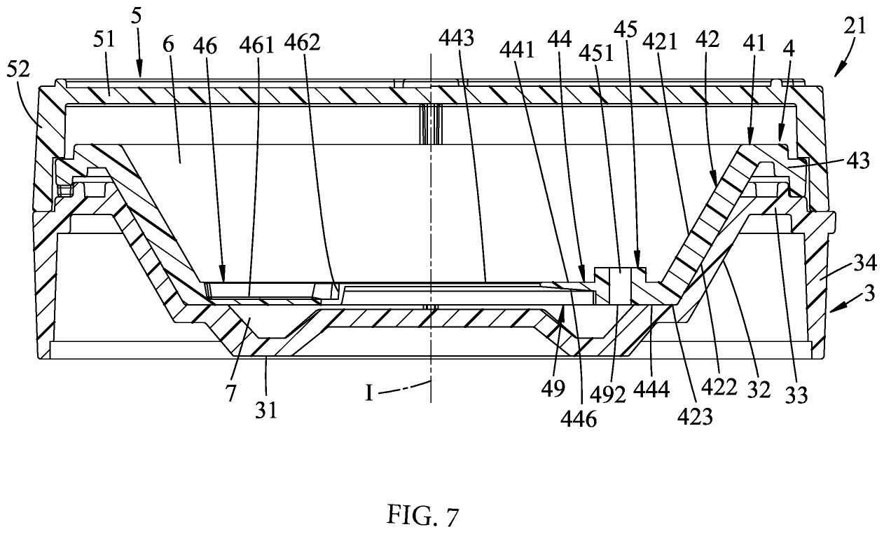

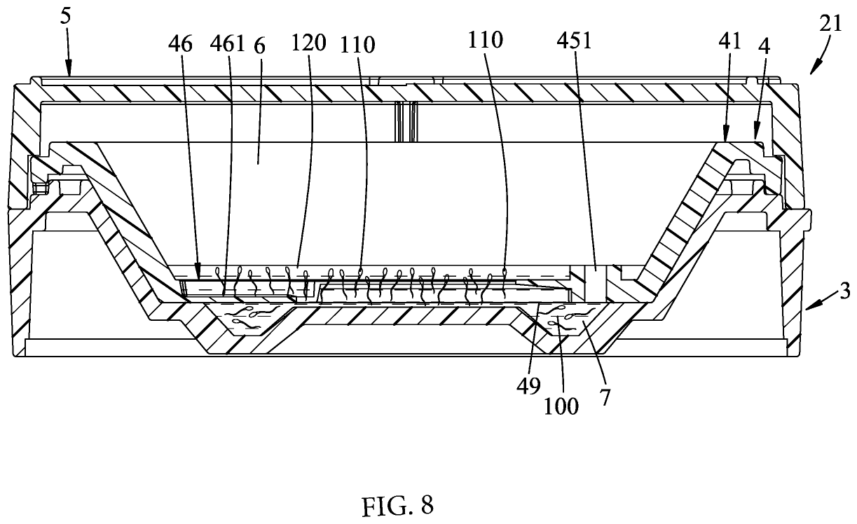

The injection portion 45 may be situated on a bottom edge of the inner sleeve surface 421. The injection portion 45 may physically extend from the inner sleeve surface 421 towards a center of the sorter 4. The base 3 and the sorter 4 may be bonded together using an adhesive substance (e.g., glue or paste). The sorter 4 may include a connecting section 445 that extends from the inner sleeve surface 421 towards the central hole 443. The injection portion 45 may extend through the connecting section 445. To use the sperm sorting device 21, referring to FIG. 4, FIG. 7, and FIG. 8, the user begins by holding an injector (not shown) that contains the semen specimen 100 and injecting the semen specimen 100 directly from the injector into the specimen chamber 7 through the injection hole 451. Following that, a sorting medium 120 is applied to the sorting filter 49 in drops so that a portion of the sorting medium 120 seeps into the specimen chamber 7 and thus forms a continuous liquid surface, wherein the sorting medium 120 may be any diluting liquid suitable for the processing of sperms. After that, the semen specimen 100 and the sorting medium 120 are allowed to stand for 3 to 60 minutes, during which time the motile sperms 110 in the semen specimen 100 that have high motility will swim upward, pass through the micropores 491 of the sorting filter 49, and end up in the sorting chamber 6 and mixed with the sorting medium 120. Lastly, the user inserts the needle of a liquid taking device (e.g., a syringe, not shown) into the position-limiting groove 46 for a liquid taking device and, with the needle pressed against the groove bottom portion 461, draws the sorting medium 120 and the motile sperms 110 with high motility by suction. During the process, not only is the needle of the liquid taking device kept from puncturing the sorting filter 49, but also the portion of the sorting medium 120 that is located between the sorting filter 49 and the annular bottom surface 442 of the liquid drawing ring 44 can be drawn out to reduce residues of the sorting medium 120, thanks to the position-limiting groove 46 for a liquid taking device being in communication with the sorting filter 49 through the communicating portion 462.

It is worth mentioning that while the sorting medium 120 and the motile sperms 110 are being drawn by suction, the spaced-apart ribs 352 on the central protruding block 35 can hinder the flow of the semen specimen 100 and thereby prevent the impurities in the semen specimen 100 from being drawn out through the sorting filter 49. The impurities may also be stopped at the junctions between the truncated cone-shaped surface 351 and the spaced-apart ribs 352 and thus prevented from being drawn out through the sorting filter 49, the objective being to enhance sorting quality.

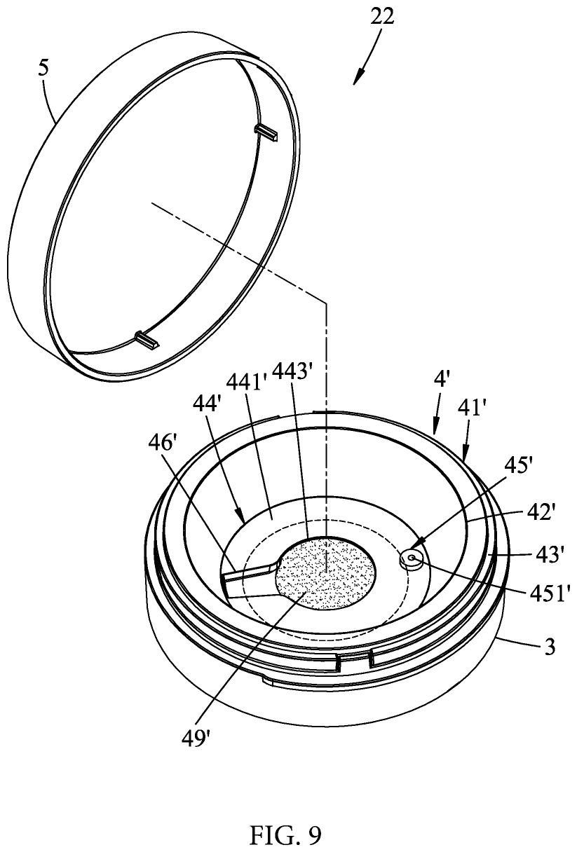

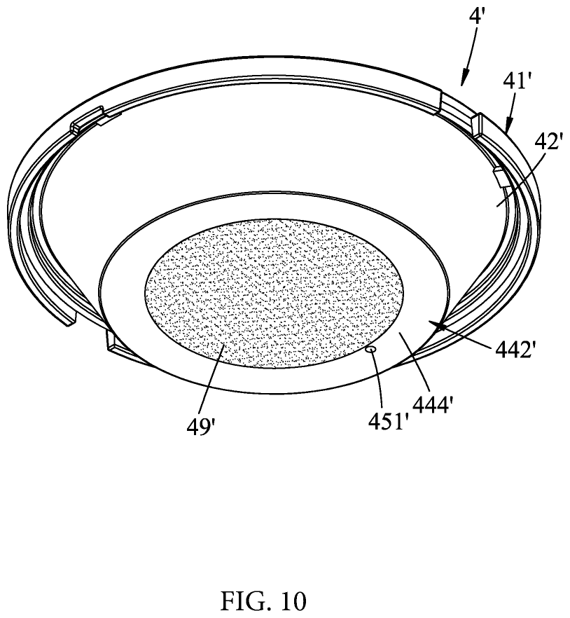

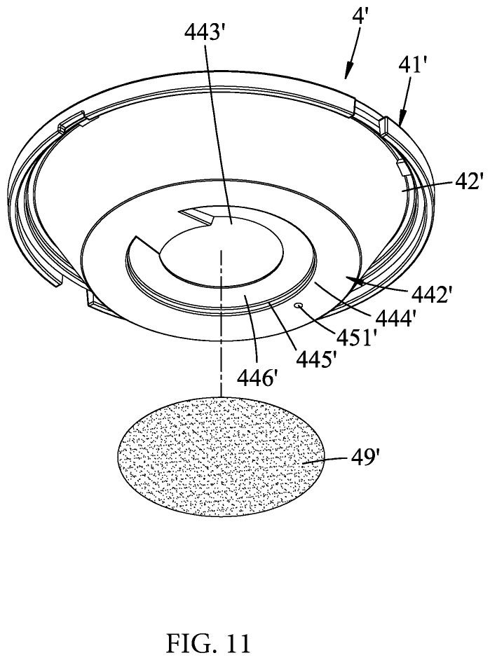

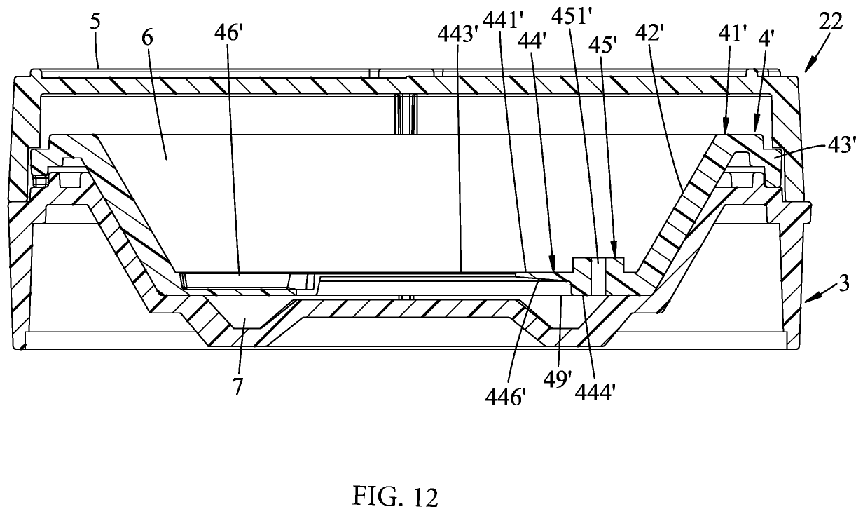

Referring to FIG. 9 to FIG. 12, the sperm sorting device 22 according to the second embodiment of the present invention includes the base 3, a sorter 4′, and the cover 5.

As in the first embodiment, the sorter 4′ includes a mounting member 41′ and a sorting filter 49′.

Like their respective counterparts in the first embodiment, the mounting member 41′ has a sleeve 42′, a protruding ring 43′, a liquid drawing ring 44′, an injection portion 45′, and a position-limiting groove 46′ for a liquid taking device; the liquid drawing ring 44′ has an annular top surface 441′, an annular bottom surface 442′, and a central hole 443′; and the annular bottom surface 442′ has an outer extension section 444′, a connecting section 445′, and an inner extension section 446′.

The sperm sorting device 22 is different from the sperm sorting device 21 in that the injection hole 451′ of the injection portion 45′ penetrates, and brings into communication, the annular top surface 441′ and the outer extension section 444′ of the annular bottom surface 442′, and that the sorting filter 49′ is fixed on the outer extension section 444′, does not extend beyond or under the injection hole 451′, and therefore need not be provided with a through hole corresponding to the injection hole 451′.

Like the sperm sorting device 21, the sperm sorting device 22 defines the specimen chamber 7 and the sorting chamber 6, which in this embodiment communicate with each other vertically in a direct manner through the injection hole 451′.

The sperm sorting device 22 can be used in the same way and produce the same effects as the sperm sorting device 21 and therefore will not be described any further.

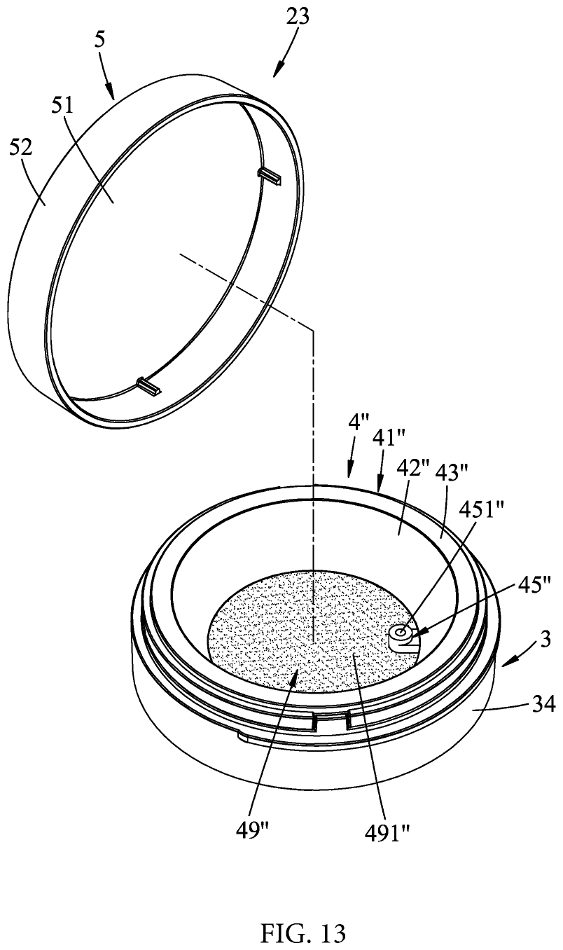

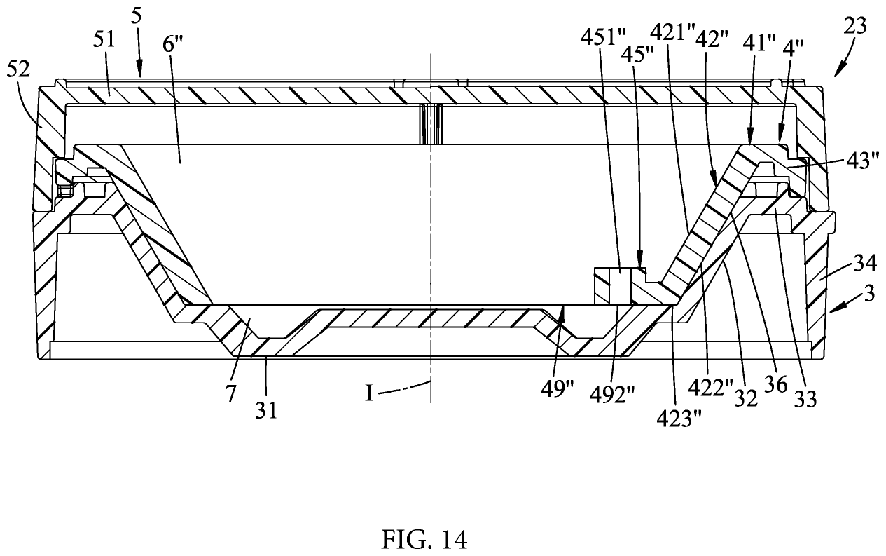

Referring to FIG. 13 and FIG. 14, the sperm sorting device 23 according to the third embodiment of the present invention includes the base 3, a sorter 4″, and the cover 5.

As in the first embodiment, the sorter 4″ includes a mounting member 41″ and a sorting filter 49″.

Like its counterpart in the first embodiment, the mounting member 41″ has a sleeve 42″ surrounding the axis I, placed in the receiving space 36, and securely coupled to the surrounding wall 32; a protruding ring 43″ extending from the top end of the sleeve 42″ in a direction away from the axis I and securely coupled to the top wall 33 of the base 3; and an injection portion 45″ extending from the bottom end of the sleeve 42″ toward the axis I.

Like its counterpart in the first embodiment, the sleeve 42″ has an inner sleeve surface 421″, an outer sleeve surface 422″, and a bottom sleeve surface 423″.

The injection portion 45″ is penetrated by an injection hole 451″ along a direction parallel to the axis I.

The sorting filter 49″ is fixed on the bottom sleeve surface 423″ of the sleeve 42″ or the bottom end of the sleeve 42″ and has a plurality of micropores 491″ and a through hole 492″ that corresponds to the injection hole 451″.

Preferably, the sorting filter 49″ is further fixed to the bottom surface of the injection portion 45″ to increase the area over which the sorting filter 49″ is fixed to the sleeve 42″ and to thereby better secure the sorting filter 49″ in place.

The sperm sorting device 23 is different from the sperm sorting device 21 in that the mounting member 41″ dispenses with the liquid drawing ring 44 and the position-limiting groove 46 for a liquid taking device (see FIG. 4), that the sorting filter 49″ is fixed on the bottom sleeve surface 423″ of the sleeve 42″, and that the sorting filter 49″, the injection portion 45″, and the inner sleeve surface 421″ jointly define a sorting chamber 6″ with an open top end.

Like the sperm sorting device 21, the sperm sorting device 23 defines the specimen chamber 7, which in this embodiment communicates with the sorting chamber 6″ vertically in a direct manner through the injection hole 451″.

It can be known from the above that the sperm sorting devices 21, 22, and 23 of the present invention have the following effects and advantages:
1. As the injection portion 45 provided on the liquid drawing ring 44 is provided with the injection hole 451, and the injection portion 45′ provided on the liquid drawing ring 44′ is provided with the injection hole 451′, the specimen chamber 7 and the sorting chamber 6 can communicate with each other vertically in a direct manner, allowing the semen specimen 100 to be injected through the injection hole 451 or 451′ into the specimen chamber 7 rapidly. The sperm sorting devices 21 and 22, therefore, can be operated with less time and effort than their prior art counterparts.
2. As the injection portion 45″ provided at the bottom end of the sleeve 42″ is provided with the injection hole 451″, the specimen chamber 7 and the sorting chamber 6″ can communicate with each other vertically in a direct manner, allowing the semen specimen to be injected through the injection hole 451″ into the specimen chamber 7 rapidly. The sperm sorting device 23, therefore, can be operated with less time and effort than its prior art counterparts.
3. The present invention does not require the flow path 15 in the conventional sperm sorting system 10 and therefore has a simpler overall structure and consequently a lower production cost than the conventional sperm sorting system 10.
4. As the present invention does not require the horizontally extending flow path 15 in the conventional sperm sorting system 10, the diameters of the sperm sorting devices 21, 22, and 23 of the invention need not be increased because of the flow path.

According to the above, the sperm sorting devices disclosed herein are indeed capable of achieving the objectives of the present invention.

The embodiments described above are only some feasible ones of the present invention and should not be construed as restrictive of the scope of the invention. Any simple equivalent change or modification based on the appended claims and the contents of this specification shall fall within the scope of the invention.

What is claimed is:

1. A sperm sorting device, suitable for selectively isolating a plurality of motile sperms from a semen specimen, the sperm sorting device comprising:
a base including a bottom wall and a surrounding wall, the surrounding wall extending from a periphery of the bottom wall toward a top end of the base and surrounding an axis, the bottom wall and the surrounding wall jointly defining a receiving space with an opening; and
a sorter securely coupled to the base and including a mounting member and a sorting filter, wherein:
the mounting member has a sleeve, a liquid drawing ring, and an injection portion, the sleeve surrounding the axis and being located in the receiving space, the liquid drawing ring extending from a bottom end of the sleeve toward the axis, the injection portion being provided on the liquid drawing ring, the injection portion being penetrated by an injection hole along a direction parallel to the axis, the sleeve having an inner sleeve surface and an outer sleeve surface surrounding the inner sleeve surface; and
the sorting filter is fixed on the sleeve such that the sorting filter, the liquid drawing ring, and the inner sleeve surface jointly define a sorting chamber with an open top end, the sorting filter having a plurality of micropores allowing passage of the motile sperms;
wherein the sorting filter, the bottom wall, and the surrounding wall jointly define a specimen chamber for receiving the semen specimen, and the injection hole brings the specimen chamber and the sorting chamber into communication with each other vertically.

2. The sperm sorting device of claim 1, wherein the base and the sorter are integrally formed as a single structure.

3. The sperm sorting device of claim 1, wherein the sleeve further has a bottom sleeve surface connecting the inner sleeve surface and the outer sleeve surface, the liquid drawing ring has an annular top surface, an annular bottom surface, and a central hole, the central hole brings the annular top surface and the annular bottom surface into communication with each other, and the annular bottom surface of the liquid drawing ring approaches the annular top surface gradually while extending from the bottom sleeve surface of the sleeve toward the axis.

4. The sperm sorting device of claim 3, wherein the annular bottom surface of the liquid drawing ring has an outer extension section extending from the bottom sleeve surface of the sleeve toward the axis, a connecting section extending from the outer extension section toward the annular top surface, and an inner extension section extending from the connecting section toward the axis, the central hole brings the annular top surface and the inner extension section of the annular bottom surface into communication with each other, the injection hole penetrates the annular top surface and the connecting section of the annular bottom surface, and the sorting filter is fixed on the outer extension section and has a through hole corresponding to the injection hole.

5. The sperm sorting device of claim 3, wherein the annular bottom surface of the liquid drawing ring has an outer extension section extending from the bottom sleeve surface of the sleeve toward the axis, a connecting section extending from the outer extension section toward the annular top surface, and an inner extension section extending from the connecting section toward the axis, the central hole brings the annular top surface and the inner extension section of the annular bottom surface into communication with each other, the injection hole penetrates the annular top surface and the outer extension section of the annular bottom surface, and the sorting filter is fixed on the outer extension section but does not extend beyond or under the injection hole.

6. The sperm sorting device of claim 3, wherein the injection portion further has a top protruding block formed on the annular top surface of the liquid drawing ring and surrounding the injection hole.

7. The sperm sorting device of claim 6, wherein the injection portion further has a bottom protruding block formed on the annular bottom surface of the liquid drawing ring and surrounding the injection hole.

8. A sperm sorting device, suitable for selectively isolating a plurality of motile sperms from a semen specimen, the sperm sorting device comprising:
a base including a bottom wall and a surrounding wall, the surrounding wall extending from a periphery of the bottom wall toward a top end of the base and surrounding an axis, the bottom wall and the surrounding wall jointly defining a receiving space with an opening; and
a sorter securely coupled to the base and including a mounting member and a sorting filter, wherein:
the mounting member has a sleeve and an injection portion, the sleeve surrounding the axis and being located in the receiving space, the injection portion extending from a bottom end of the sleeve toward the axis, the injection portion being penetrated by an injection hole along a direction parallel to the axis, the sleeve having an inner sleeve surface and an outer sleeve surface surrounding the inner sleeve surface; and
the sorting filter is fixed on the bottom end of the sleeve such that the sorting filter, the injection portion, and the inner sleeve surface jointly define a sorting chamber with an open top end, the sorting filter having a plurality of micropores allowing passage of the motile sperms and a through hole corresponding to the injection hole;
wherein the sorting filter, the bottom wall, and the surrounding wall jointly define a specimen chamber for receiving the semen specimen, and the injection hole brings the specimen chamber and the sorting chamber into communication with each other vertically.

9. The sperm sorting device of claim 8, wherein the sleeve further has a bottom sleeve surface connecting the inner sleeve surface and the outer sleeve surface, and the sorting filter is fixed on the bottom sleeve surface of the sleeve.

10. The sperm sorting device of claim 9, wherein the sorting filter is further fixed to a bottom surface of the injection portion.

11. A sperm sorting device comprising:
a base including a bottom wall and a surrounding wall, the surrounding wall extending from a periphery of the bottom wall toward a top end of the base and surrounding an axis, the bottom wall and the surrounding wall jointly defining a receiving space; and
a sorter, the sorter comprising:
a protruding ring located on a periphery of the sorter, the protruding ring including:
an inner sleeve surface that extends towards a bottom of the sorter; and
a central hole for a sorting filter that spans the central hole and filters the semen specimen; and
an injection portion configured to join a sorting space above the sorter to the receiving space, the injection portion situated on a bottom edge of the inner sleeve surface.

12. The sperm sorting device of claim 11, wherein the sorting filter includes a through hole that aligns with the injection portion.

13. The sperm sorting device of claim 11, wherein the sorting filter does not extend over the injection portion.

14. The sperm sorting device of claim 11, wherein the injection portion physically extends from the inner sleeve surface toward a center of the sorter.

15. The sperm sorting device of claim 11, wherein the base and sorter are bonded together using an adhesive substance.

16. The sperm sorting device of claim 11, wherein the injection portion is located above the receiving space.

17. The sperm sorting device of claim 11, wherein the sorter includes a connecting section that extends from the inner sleeve surface towards the central hole, wherein the injection portion extends through the connecting section.

18. A method for isolating motile sperm from a semen specimen, the method comprising:
injecting the semen specimen through an injection portion of a sperm sorting device into a receiving space of the sperm sorting device, the sperm sorting device comprising:
a base including a bottom wall and a surrounding wall that jointly define a receiving space with an opening; and
a sorter including:
a protruding ring located on a periphery of the sorter, the protruding ring including:
a sleeve that extends towards a bottom of the sorter; and
a central hole for a sorting filter that spans the central hole and filters the semen specimen;
a liquid drawing ring extended from a bottom of the sleeve and including a position-limiting groove; and
the injection portion configured to join a sorting space above the sorter to the receiving space, the injection portion situated on a bottom edge of the inner sleeve surface; and
applying a sorting medium to the sorting filter in drops; and
drawing the sorting medium and isolated motile sperms from the position-limiting groove using a liquid taking device.

19. The method of claim 18, further comprising:
allowing the semen specimen and sorting medium to stand for 30 to 60 minutes before drawing the sorting medium and isolated motile sperms.

20. The method of claim 18, wherein the sorting medium is a diluting liquid suitable for processing sperms.
